(19) United States
(12) Patent Application Publication
(10) Pub. No.: US 2020/0276087 A1
(43) Pub. Date: Sep. 3, 2020

(54) PHARMACEUTICAL MANUFACTURING INSTALLATION AND METHOD OF MANUFACTURING OF A PHARMACEUTICAL PRODUCT

(71) Applicant: HOFFMANN-LA ROCHE INC., Little Falls, NJ (US)

(72) Inventors: Juraj SIBIK, Basel (CH); Pascal CHALUS, Basel (CH)

(21) Appl. No.: 16/648,873

(22) PCT Filed: Sep. 20, 2018
(86) PCT No.: PCT/EP2018/075415

§ 371 Date: Mar. 19, 2020

(30) Foreign Application Priority Data
Sep. 21, 2017 (EP) 17192423.6

(51) Int. Cl. A61J 3/10; G01N 27/22; G01N 33/15; G01N 21/95; B30B 11/00

(52) U.S. Cl. CPC A61J 3/10; G01N 27/221; B30B 11/005; G01N 21/9508; G01N 33/15

(57) ABSTRACT

A pharmaceutical manufacturing installation, including a processing equipment arranged to provide a pharmaceutical substance in a solid state and a solid fraction sensor is disclosed. The solid fraction sensor having a first conductor element, a second conductor element, an operation space, an energy source arranged to generate an electric field in the operation space by means of the first conductor element and the second conductor element, and a controller adapted to determine a capacitance between the first and second conductor element of the pharmaceutical substance in the solid state located in the operation space. The solid fraction sensor is arranged to receive the pharmaceutical substance in its solid state into the operation space by the processing equipment.

### TECHNICAL FIELD

The present invention relates to a pharmaceutical manufacturing installation comprising a processing equipment arranged to provide a pharmaceutical substance in a solid state and a solid fraction sensor, and, furthermore, the invention relates to a method of manufacturing of a pharmaceutical product.

### BACKGROUND ART

Porosity of solids has a tremendous effect on their mechanical properties and is hence of importance in many industries, including pharmaceutical, chemical or food industry. In pharmaceutical manufacturing, porosity of the intermediates influences also the porosity of the final solid dosage forms, while the porosity of the final dosage forms influence their disintegration and dissolution behaviour. Hence, porosity of the intermediates and final dosage forms plays an important role in the bioavailability of pharmaceutical products.

The intermediate porosity is of particular importance in dry granulation of powder mixtures via roller compaction and in tablet pressing. In roller compaction, the powder mixture is first pressed into a ribbon using two spinning rolls and the ribbon is then milled into granules. Using too small compaction force during the roller compaction can result in fragile granules and high content of small granules, with only limited improvement in the flowability and prevention of segregation in comparison to the input powder mixture. On the other hand, too large compaction force would take away significant part of compressibility of the powder and prohibit further pressing into tablet. Knowledge of ribbon porosity can serve as a good indication for both granule size and tablet mechanical properties of a target pharmaceutical sample. In tablets, too high porosity will likely result in chipping and breaking of the tablet, while too low porosity may negatively affect the release of the drug substance from the tablet.

Commonly, in pharmaceutical manufacturing installations, the porosity of solid state intermediates and final products is determined by off-line analysis. When the true density is known, the bulk porosity can be determined by simple measurements of weight and bulk volume. For more accurate determination of volume for a sample with uneven thickness one often uses surface scanning laser confocal displacement meter. On the other hand, technologies like pycnometry can provide absolute measure of porosity and pore distribution without any prior knowledge, although at a higher labour cost.

Further, there is an on-going search for pharmaceutical manufacturing installations with suitable process analytical technologies (PAT) around manufacturing pharmaceutical products and their intermediates. In particular, it is aimed to achieve processing without any interruption such that the above off-line analysis typically is not appropriate. In this context, some earlier proposals considered utilization of NIR spectroscopy, a common PAT tool which is sensitive to both chemical and physical properties of the sample. However, NIR provides an indirect measure of porosity based on a somewhat impractical multivariate calibration and it is not trivial to isolate the undesired chemical and other physical effects in the porosity prediction. Terahertz spectroscopy provides a more accurate and easier to calibrate alternative, but is still relatively new to pharmaceutical industry and requires further design for implementation as PAT tool. Recently, a novel low-cost measurement based on thermal imaging has been proposed as a solution for ribbon porosity analysis during roller compaction. It is however suitable only for the ribbons of sufficient quality and it requires careful consideration of environmental effects. All of the aforementioned techniques are however still comparably difficult to adapt for inline/online automated measurements in pharmaceutical manufacturing installations that could be used as process analytical technology.

Therefore, an object of the invention is to further develop a pharmaceutical manufacturing installation or method to provide for suitable inline/online automated measurements.

### DISCLOSURE OF THE INVENTION

According to the invention this need is settled by a pharmaceutical manufacturing installation with the features of independent claim 1 and by method of manufacturing a pharmaceutical product with the features of independent claim 8. Preferred embodiments are subject of the dependent claims.

In particular, in one aspect of the invention, a pharmaceutical manufacturing installation comprises a processing equipment arranged to provide a pharmaceutical substance in a solid state and a solid fraction sensor. The solid fraction sensor has a first conductor element, a second conductor element, an operation space, an energy source arranged to generate an electric field in the operation space by means of the first conductor element and the second conductor element, and a controller adapted to determine a capacitance between the first and second conductor elements with the pharmaceutical substance in the solid state located in the operation space. The solid fraction sensor is arranged to receive the pharmaceutical substance in its solid state into the operation space by the processing equipment.

The term “drug” as used herein below can relate to a therapeutically active agent, also commonly called active pharmaceutical ingredient (API), as well as to a combination of plural such therapeutically active substances. The term also encompasses diagnostic or imaging agents, like for example contrast agents (e.g. MRI contrast agents), tracers (e.g. PET tracers) and hormones, that need to be administered in liquid form to a patient.

The term “pharmaceutical substance” as used herein can relate to a drug as defined above formulated or reconstituted in a form that is suitable for administration to the patient. For example, besides the drug, a drug or pharmaceutical substance may additionally comprise an excipient and/or other auxiliary ingredients. The pharmaceutical substance can also be a formulation only including one or more excipients and/or other auxiliary ingredients.

The term “drug product” as used herein can relate to a finished end product, comprising a pharmaceutical substance or a plurality of pharmaceutical substances. In particular, a drug product may be a ready to use product having the pharmaceutical substance in an appropriate dosage and/or in an appropriate form for administration. For example, a drug product may include an administration device such as a prefilled syringe or the like. The reference can particularly be similar to the target pharmaceutical sample, wherein, besides the essentially same dielectric properties, the samples can have the same API and/or the essentially same chemistry and/or the essentially same composition.

With the use of solid fraction sensor in the pharmaceutical manufacturing installation according to the invention, it is possible to measure the change in the capacitance of the solid fraction sensor induced by the presence of the pharmaceutical substance. The knowledge of the solid fraction sensor geometry, the pharmaceutical substance geometry and the capacitance change can be used to extract the real part of a dielectric permittivity of the pharmaceutical substance. The measured dielectric permittivity can be calibrated with respect to the pharmaceutical substance's solid fraction allowing a current dielectric sensor to be used as a solid fraction sensor inside a pharmaceutical manufacturing installation. Like this, the solid fraction of the manufactures pharmaceutical substance can efficiently and accurately be determined online or inline.

This setup offers significant practical advantages in comparison to state of the art methods, namely: it is applicable both off-line/at-line as well as inline/online measurement; no electrical contact with the target pharmaceutical sample is needed; and a sensitivity of around less than 3% absolute solid fraction deviation can be achieved. In addition, the possible read-out time can be less than 10 ms, which is fast enough for the desired inline/online application. The use of the current solid fraction sensor can show a good linearity in the target range of interest between 50 and 100% solid fraction for pharmaceutical intermediates and products, such as ribbons and tablets. Furthermore, the measurement can be robust, because it shows low impact of e.g. a product lamination or a product fractionation of the target pharmaceutical sample. Still further, in the setup according to the invention it can be prevented that an electrical contact is required between the pharmaceutical substance and the first or second conductor element.

For an appropriate functioning, the solid fraction sensor preferably is electromagnetically shielded. Like this, disturbances induced by other parts of the manufacturing installation or still other things can be prevented or minimized.

In a preferred embodiment, the processing equipment of the pharmaceutical manufacturing installation comprises a punch arrangement adapted to generate a tablet of the pharmaceutical substance. Thereby, one of the first conductor element and the second conductor element preferably is a part of the punch arrangement of the processing equipment or of a chute or outlet of the processing equipment. Such implementation of one conductor element by one part of the punch arrangement thereof allows for an efficient integration of the solid fraction sensor into the processing equipment. Like this, a compact and efficient arrangement can be achieved.

In another preferred embodiment, the processing equipment comprises two rotatable rolls and a compacting space in between the rolls, wherein the compacting space has a powder inlet zone and a ribbon outlet zone, and the solid fraction sensor is arranged adjacent to the ribbon outlet zone of the compacting space such that a ribbon generated by the two rolls exiting the compacting space is forwarded into the operation space of the solid fraction sensor. Such arrangement allows for efficiently providing information about the solid fraction of the ribbon.

Thereby, the second conductor element preferably is formed as a ribbon support adapted to guide a ribbon exiting the compacting space. Alternatively, one of the two rolls preferably is the second conductor element, wherein the first conductor element can be bent to correspond to the outer surface of the one of the two rolls. Such embodiment of the second conductor element allows for efficiently integrating the solid fraction sensor into the roller compactor pharmaceutical manufacturing installation.

In a preferred embodiment, the controller of the solid fraction sensor comprises calibration data of a reference pharmaceutical substance having the essentially same dielectric properties as the pharmaceutical substance in its solid state, the calibration data of the controller of the solid fraction sensor comprises composition data about the composition of the reference pharmaceutical substance and thickness data about the thickness of the reference pharmaceutical substance, the controller of the solid fraction sensor is adapted to convert the calibration data and the determined capacitance into solid fraction data of the pharmaceutical substance in its solid state, and the controller is adapted to generate a solid fraction signal representing the solid fraction data. In such an embodiment, the solid fraction can automatically be evaluated in a particularly efficient and accurate manner.

The solid fraction signal can be in any suitable form such that information about the solid fraction, i.e. the solid fraction data, is represented. For example, the signal can be an electrical signal, a ultrasonic or other acoustic signal, a (laser) light signal or the like.

Preferably, the controller of the solid fraction sensor has a data storage in which the calibration data is stored. The data storage can be any suitable permanent or volatile data storage such as, e.g., a flash memory, a hard disk, a memory chip, an external storage or cloud storage, or the like.

The calibration data preferably comprises permittivity of the reference pharmaceutical substance and a solid fraction ratio of the reference pharmaceutical substance. Generally, permittivity (ε) or dielectric permittivity can be a measure of resistance that is encountered when forming an electric field in a medium. Relative permittivity can be the factor by which an electric field between charges is decreased relative to vacuum. More specifically, ε can describe the amount of charge needed to generate one unit of electric flux in the medium. Accordingly, a charge will yield more electric flux in a medium with low ε than in a medium with high ε. Thus, ε is the measure of a material's ability to resist an electric field rather than its ability to permit it. Typically, ε is specified in Farad per meter (F/m). Such information allows an efficient and accurate evaluation of the solid fraction of the target pharmaceutical sample.

Particularly, the calibration data preferably comprises pairs of permittivity and corresponding solid fraction ratio. With such pairs, the permittivity and solid fraction ration can efficiently be interrelated. In particular, the information about the composition of the reference pharmaceutical substance and about the thickness of the reference pharmaceutical substance preferably is a calibration curve. Such a calibration curve allows for an efficient and reproducible evaluation.

Preferably, the energy source of the solid fraction sensor is connected to at least one of the first conductor element and the second conductor element. This allows for an efficient implementation of the sensor. In the same context, the controller preferably is adapted to adjust a strength of the electric field in the operation space.

The first and second conductors of the solid fraction sensor can be made of any suitable conductive material. They can further have any predefined shape or geometry. However, in a preferred and comparably simple embodiment, the first conductor element of the solid fraction sensor and the second conductor element of the solid fraction sensor are metallic and plate-like shaped. The term “plate-like” as used herein can relate to a plate being straight, even or bent. It can also relate to a plane, structured or uneven plate. Such plates allow for easily defining the operation space in between themselves which can efficiently be evaluated since the well defined and eventually simple geometry. In a specific example, the first conductor element can be provided in form of a roll of a roller compaction for pressing a power mixture of the target pharmaceutical sample into a ribbon, while the second conductor element can be a curved segment, which limits the operation space between both conductor elements.

The controller of the solid fraction sensor can be adapted in any suitable manner for determining the capacitance. For example, it can involve a time based determination in which, typically, an unknown capacitance is used to modify an oscillator circuit frequency. Or, it can involve a bridge determination in which two voltage dividers are compared wherein one path is known and the other one comprises the unknown capacitance.

In a preferred embodiment of the solid fraction sensor, the controller of the solid fraction sensor is adapted to determine the capacitance by a capacitance-to-digital conversion (CDC) and, more specifically, it can be adapted to apply sigma-delta modulation to determine the capacitance.

In another embodiment of the solid fraction sensor, is adapted to measure a discharge time and to determine the capacitance by using the measured discharge time. For example, the solid fraction sensor can be implemented as or comprise a PICO-CAP converter.

In still another preferred embodiment of the solid fraction sensor, the controller is adapted to determine the capacitance by using a charge-balancing circuit or method.

For adjusting the gap between the first and the second conductor element or for adjusting the size of the operation space, respectively, the solid fraction sensor preferably comprises a displacement structure, wherein the at least one of the first conductor element of the solid fraction sensor and the second conductor element of the solid fraction sensor is mounted to the displacement structure such that the first conductor element of the solid fraction sensor and the second conductor element of the solid fraction sensor are movable relative to each other. The displacement structure allows the adjustment of an air-gap in the operation space to a minimum such that the accuracy of the solid fraction determination can be increased or optimized.

The operation space of the solid fraction sensor can be a space in which the first and second conductor elements may generate an electric field. For example, the first and second conductor elements may be positioned aside each other such that the operation space is located above or below the two conductor elements where the electric field can be generated. However, preferably, the solid fraction sensor is embodied such that the operation space of the solid fraction sensor is a gap separating the first conductor element and the second conductor element. Such a gap allows for well defining the operation space which makes the determination of the capacitance comparable simple and efficient.

When determining the solid fraction of the pharmaceutical substance, in general, any geometrical difference, composition difference and moisture content difference between the reference pharmaceutical substance and the pharmaceutical substance should be accounted for in order to achieve a high accuracy. For example, surface pattern, e.g. caused by ribbons produced with patterned rolls while the reference pharmaceutical substance may be produced without, may occur which can influence the accuracy of the solid fraction determination. Non-variable differences can be accounted for by correction of the measured signal prior comparison with the calibration curve or calibration data. One option is to include as many such dependencies in the multi-variate calibration curve or calibration data as feasible. However, this could be comparably cumbersome as it might cause and extensive calibration requirement.

For those properties that are more or less constant such as, e.g., moisture content, some of the dimensions and the like, the accuracy lowering effects may be reduced by choosing a suitable reference pharmaceutical substance that matches the properties of the pharmaceutical sample and measured at operating conditions. When this is impractical, it might be tried to account for them in the evaluation of the reference pharmaceutical substance such as, e.g., surface pattern can be accounted for instead of forcing the reference pharmaceutical substance having the same surface pattern. When the sample properties varies it might be beneficial to provide active correction by having independent measure of the variable properties. Once such properties are measured, they can be accounted for numerically instead of having multi-variate calibration.

In this context, the pharmaceutical manufacturing installation preferably comprises a thickness measuring unit adapted to measure a thickness of the pharmaceutical substance in its solid state, preferably, when positioned in the operation space of the solid fraction sensor. The thickness measuring unit can be any suitable measurement arrangement such as an electrical, mechanical, optical, acoustic or combined sensor. However, preferably, the thickness measuring sensor comprises a distance capacitance sensor. Such arrangement allows for determining the thickness of the target pharmaceutical sample by the same or similar means of principles applied for determining the capacitance.

Preferably, at least one of the first conductor element of the solid fraction sensor and the second conductor element of the solid fraction sensor is equipped with an insulating layer towards the operation space. Such insulating layer may allow for minimizing effects of parasitic resistivity of the target pharmaceutical sample on the measurement. It may further help to increase the lifetime of the respective conductor element. Also, it may help to prevent contamination of the target pharmaceutical sample. Still further, it may prevent or reduce dust build up on the sensor. Finally, it may also allow for easier cleaning of the sensor and particularly its conductor elements.

Preferably, the solid fraction sensor comprises a reference third conductor element and a reference fourth conductor element together establishing a reference capacitor, wherein the controller of the solid fraction sensor is adapted to being responsive to a difference between an output of a measuring capacitor established by the first conductor element and the second conductor element and an output of the reference capacitor. By providing such reference capacitor the influence of the environmental and operating conditions, such as temperature, humidity or the like, on the sample measurement can be reduced or minimized. In particular, it may allow compensation in situation where the calibration curve does not correspond to the operating conditions and, thus, the sensing may be inaccurate.

Preferably, the first conductor element and/or the second conductor element of the solid fraction sensor has a surface area adjacent to the operation space in a range of between 1 mm² and 10′000 mm² or preferably between 10 mm² and 1′000 mm².

Preferably, the solid fraction sensor establishes a sensor circuit which operates with a dynamic range of 0 Picofarad (pF) to 1′000 pF, preferably of 0 pF to 100 pF and particularly of 0 pF to 10 pF. The sensor circuit preferably operates with a sensitivity of less than 1′000 Femtofarad (fF), preferably less than 100 fF and particularly less than 10 fF.

Another aspect of the invention relates to a method of manufacturing a pharmaceutical product comprising the steps of: providing a pharmaceutical substance in a solid state; positioning the pharmaceutical substance in its solid state in an operation space in which an electric field is generated by means of a first conductor element and a second conductor element; determining a capacitance of the pharmaceutical substance in its solid state located in the operation space; and converting the determined capacitance together with information about a composition of a reference pharmaceutical substance having the essentially same dielectric properties as the pharmaceutical substance in its solid state and about a thickness of the reference pharmaceutical substance into a solid fraction of the pharmaceutical substance in its solid state.

The reference pharmaceutical substance can particularly be similar to the pharmaceutical substance wherein, besides the essentially same dielectric properties, the substances can have the same API and/or the essentially same chemistry and/or the essentially same composition.

The method according to the invention and its preferred embodiments described below allow for efficiently achieving the effects and benefits described above in connection with the manufacturing installation according to the invention and the embodiments thereof.

In one preferred embodiment, the pharmaceutical substance in its solid state is bounded. Such a bounded pharmaceutical substance can be a compressed substance such as a tablet, or a ribbon which is further processed to granules or the like, or otherwise bounded such as by lyophilisation. In another preferred embodiment, the pharmaceutical substance in its solid state is unbounded. Such a pharmaceutical substance can, e.g., be a lyophilized powder, any loose powdered material or the like.

Advantageously, the method comprises adjusting a strength of the electric field in the operation space.

Preferably, the information about the composition of the reference pharmaceutical substance comprises permittivity of the reference pharmaceutical substance and a solid fraction ratio of the reference pharmaceutical substance. Such information allow an efficient and accurate evaluation of the solid fraction of the pharmaceutical substance.

Thereby, the information about the composition of the reference pharmaceutical substance and about the thickness of the reference pharmaceutical substance preferably comprises pairs of permittivity and corresponding solid fraction ratio. With such pairs, the permittivity and solid fraction ratio can efficiently be interrelated. In particular, the information about the composition of the reference pharmaceutical substance and about the thickness of the reference pharmaceutical substance preferably is a calibration curve. Such a calibration curve allows for an efficient and reproducible evaluation.

In some applications of the method according to the invention, it can be advantageous to determine the capacitance by a capacitance-to-digital conversion, particularly, by applying a sigma-delta modulation. Like this, a Capacitance to Digital Converter (CDC) or sigma-delta CDC can be involved. With a sigma-delta CDC the method can be realised comparably inexpensive and can have a strong potential as a process analytical technology (PAT) in the pharmaceutical industry.

In other applications, it can be advantageous to measure a discharge time and to determine the capacitance by using the measured discharge time. For example, the measurement of the discharge time can be provided by a PICO CAP converter. Such technique can particularly provide for a suitable accuracy of the capacitance determination.

In still other applications, it can be beneficial to use a charge-balancing circuit to measure the capacitance. Such capacitance measurement can be suitable accurate and fast to be implemented online in a pharmaceutical manufacturing process.

Preferably, the at least one of the first conductor element and the second conductor element is displaced to adjust the operation space. Like this, for example, it can be achieved that the conductor elements preferably slightly contact an object arranged in the operation space. Particularly, the method can comprise a step of adjusting a distance between the first conductor element and the second conductor element such that the first conductor element and the second conductor element contact the pharmaceutical substance in its solid state when positioned in the operation space. Thereby, the occurrence of free space between the conductor elements and the object can be reduced or minimized such that the accuracy of the solid fraction determination can be increased or optimized, since best results of the capacitance measurement may be achieved, when an air gap between the target pharmaceutical sample and one of the first and/or second conductor element is as small as possible.

For an accurate evaluation of the solid fraction, it can be beneficial to further measure a thickness of the target pharmaceutical sample positioned in the operation space.

In a preferred embodiment, the method according to the invention further comprises: positioning the pharmaceutical substance in its solid state in a further operation space of the same or a further solid fraction sensor having a further first conductor element, a further second conductor element, the further operation space and a further energy source arranged to generate an electric field in the further operation space by means of the further first conductor element and the further second conductor element; determining a further capacitance of the pharmaceutical substance in its solid state located in the further operation space; converting the determined further capacitance together with the information about the composition of the reference pharmaceutical substance and about the thickness of the reference pharmaceutical substance into a further solid fraction of the pharmaceutical substance in its solid state, and determining a solid fraction distribution of the solid fraction of the pharmaceutical substance in its solid state and the further solid fraction of the pharmaceutical substance in its solid state. Particularly, when comparably large and/or comparably inhomogeneous pharmaceutical substances are involved, such determination of the solid fraction distribution can be beneficial for achieving a complete or sufficient evaluation of the pharmaceutical substance.

Thereby, the operation space and the further operation space preferably are positioned neighbouring each other. Like this, the solid fraction distribution can be determined by two adjacent capacitors established by the neighbouring operation spaces such that different parts of the pharmaceutical substance can be involved. Also, the solid fraction distribution can be determined by a multi operation space array employing the principles of electrical capacitance tomography.

In a further preferred embodiment, the method according to the invention comprises: positioning the pharmaceutical substance in its solid state in a reference operation space of a reference solid fraction sensor having a reference first conductor element, a reference second conductor element, the reference operation space and a reference energy source arranged to generate an electric field in the reference operation space by means of the reference first conductor element and the reference second conductor element; determining a reference capacitance of the pharmaceutical substance in its solid state located in the reference operation space; converting the determined reference capacitance together with the information about the composition of the reference pharmaceutical substance and about the thickness of the reference pharmaceutical substance into a reference solid fraction of the pharmaceutical substance in its solid state; and comparing the solid fraction of the pharmaceutical substance in its solid state to the reference solid fraction of the pharmaceutical substance in its solid state. Like this, the quality and accuracy of the sensing procedure can be increased.

### BRIEF DESCRIPTION OF THE DRAWINGS

The pharmaceutical manufacturing installation and the method of manufacturing a pharmaceutical product according to the invention are described in more detail herein below by way of exemplary embodiments and with reference to the attached drawings, in which:

FIG. 1a,b,c shows an arrangement of a capacitor completely filled with a dielectric, a capacitor partially filled with a dielectric and an equivalent circuit diagram for a theoretical evaluation;

FIG. 2 shows an embodiment of the pharmaceutical manufacturing installation according to the invention for the use with a pharmaceutical substance in form of a tablet;

FIG. 3a,b,c shows three further embodiments of the pharmaceutical manufacturing installation for the use with a pharmaceutical substance in form of a ribbon compressed out of a powder;

FIG. 4 shows an example of a calibration curve.

### DESCRIPTION OF EMBODIMENTS

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

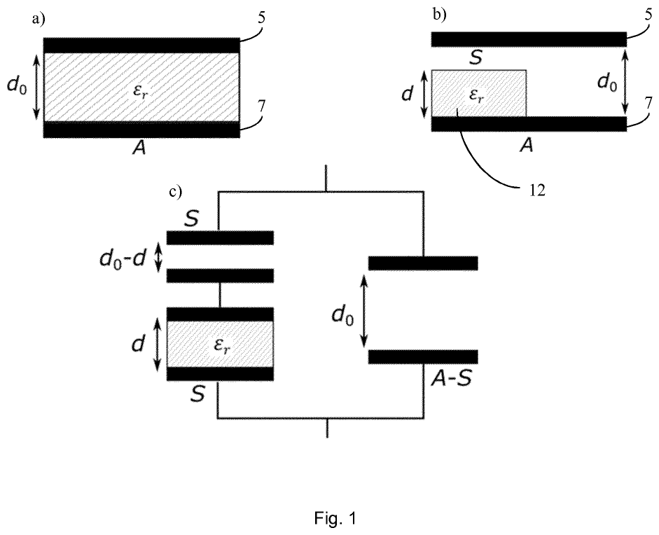

By reference to FIG. 1a,b,c a theoretical evaluation of capacitive sensing is illustrated. Capacitive sensing is a non-contact sensing widely used in many industries, including automotive, oil and gas, medical diagnostics or consumer electronics, and pharmaceutical manufacturing. In general, capacitive sensing is applicable to both conductors and non-conductors. It finds typical use as proximity and displacement sensors. Capacitive sensors are rather inexpensive, especially in comparison to spectroscopy systems, and their simple electronic nature makes them adept for online/inline implementation in manufacturing processes.

Capacitive sensing is also suitable to characterize non-conductive material properties, i.e. dielectrics. Material passing through the gap of the capacitive sensor changes the capacitance of the sensor. When the gap in the capacitor is kept constant, the sensor output will be linked to the change in the thickness, density or composition of the material. If two of these properties are kept constant, the third can be deducted from the measurement. Thus, having a material of homogeneous composition and thickness, its density can be deducted from the sensor output. With a simple calibration, this can be converted into the porosity of the material.

FIG. 1a shows an arrangement of a capacitor completely filled with a dielectric 12 between a first conductor element 5—in the following also called electrode 5—and a second conductor element 7—in the following also called electrode 7. Both electrodes 5, 7 have the same surface size A like the dielectric between them, which dielectric has a thickness of d₀ and a permittivity of εᵣ. The capacitance C of simple parallel plates is governed by

$$C = \frac{{ɛ}_{0} {ɛ}_{r} A}{{d}_{0}} \qquad ( Eq . 1 )$$

Here ε₀ is the permittivity of vacuum (ε₀=8.85149 pF/m), εᵣ is the relative permittivity of a material between electrodes (εᵣ=1 for air), A is the surface area of the electrodes and d₀ is the distance between the electrodes 5, 7. In order to evaluate the relative permittivity of the material of interest, namely the dielectric, one would normally first obtain the capacitance C₀ of empty sensor and capacitance C of sensor fully filled with the material of interest. From the difference between these two, ΔC=C−C₀, one can express the relative permittivity of the material as:

$${ɛ}_{r} = 1 + \frac{Δ {Cd}_{0}}{{ɛ}_{0} A} \qquad ( Eq . 2 )$$

If the dielectric sample fills the full thickness of the sensor but does not cover the full area A (not shown), the resulting sensor can be represented by two capacitors in series, one filled with vacuum (air) and another with the sample. The change in capacity is influenced only by the covered surface area S (corresponding to sample surface area), hence one can simply adapt Eq. 2 as

$${ɛ}_{r} = 1 + \frac{Δ {Cd}_{0}}{{ɛ}_{0} S} \qquad ( Eq . 3 )$$

It is to note that, within the approximation of homogeneous electric field between the electrodes (i.e. far from the sensor edges), the position of the sample on the electrode does not matter.

A further generalization is necessary in case that the sample does not fill the full thickness of the sensor, as shown in FIG. 1b. The resulting air gap can be represented by two capacitors in series, one filled with air and another with the material of interest, see also FIG. 1c with the corresponding equivalent circuit diagram. It is beneficial to define thickness fraction, where d corresponds to the sample thickness. The relative permittivity of the material with thickness d<d₀ can be then expressed as

$${ɛ}_{r} = 1 + {( \frac{d}{{d}_{0}} \frac{{ɛ}_{0} S}{Δ {Cd}_{0}} + \frac{d}{{d}_{0}} - 1 )}^{- 1} \qquad ( Eq . 4 )$$

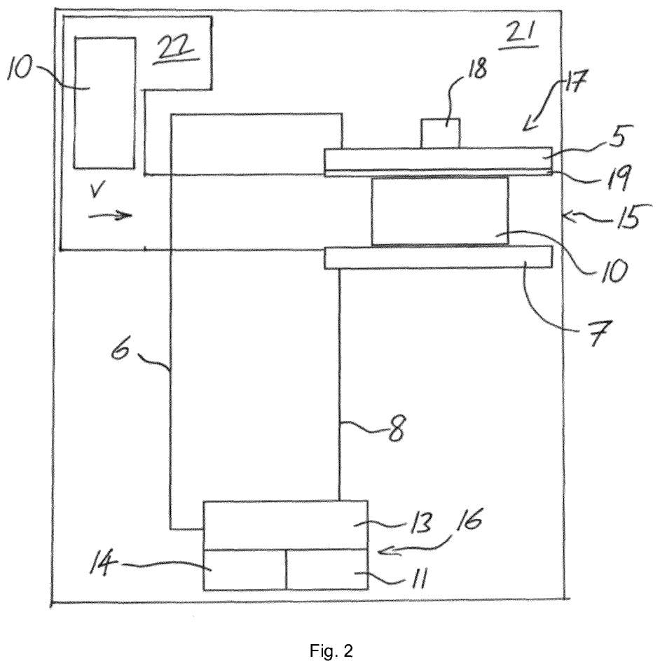

With a view to FIG. 2 the following sensitivity estimation can be determined. A change in sample porosity will result in change in the sample relative permittivity. The non-trivial part is finding a suitable circuitry that allows sensitive enough detection of changes in the relative permittivity. For example, consider a sensor with 3 mm gap between the electrodes 5 and 7 being filled with a 10 mm diameter and 2 mm thick non-porous tablet made of microcrystalline cellulose, which has relative permittivity εᵣ=5.6 at 58% relative humidity and 22° C. Using Eq. 1, the increase in the sensor capacitance in the presence of tablet would be approximately 1.6 pF. If a drop in the solid fraction results in a drop of relative permittivity by e.g. 1%, the expected drop in the sensor capacitance would be approx. 20 fF. Hence, the sensing circuit has to be able to detect the capacitance with a few fF accuracy for any practical application as a porosity sensor for solid dosage forms.

FIG. 2 shows as a unit a pharmaceutical installation according to the invention comprising a processing equipment 21 providing a pharmaceutical substance 10 in a solid state and comprising a solid fraction sensor 17. Additionally, a punch arrangement 22 for generating tablets 10 out of the pharmaceutical substance is part of this unit.

In FIG. 2 the pharmaceutical substance is a tablet 10, which is located in the operation space 15 between a first conductor element 5 and a second conductor element 7 of the solid fraction sensor 17. An energy source 13 is connected to the first and second conductor element 5, 7 via a cable 6 respectively via a cable 8. A controller 11 is adapted to adjust a strength of the electric field in the operation space 15 and furthermore, the controller 11 has a data storage 14 in which calibration data is stored. The data storage 14 can be any suitable permanent or volatile data storage such as, e.g., a flash memory, a hard disk, a memory chip, an external storage or cloud storage, or the like.

According to the invention the controller 11 can be adapted to determine the capacitance by a capacitance-to-digital conversion based on the charge-balancing method in combination with the known sigma-delta modulation.

Alternatively, the solid fraction sensor 17 adapted to measure a discharge time and to determine the capacitance by using the measured discharge time, wherein the solid fraction sensor 17 can be implemented as or comprise a PICO-CAP converter.

Furthermore, the solid fraction sensor comprises a displacement structure 18, wherein at least one of the first conductor element 5 and the second conductor element 7 is mounted to the displacement structure 18 such that the first conductor element 5 and the second conductor element 7 are movable relative to each other. By moving the first conductor element 5 and the second conductor 7 relative to each other, the size of the operation space 15 can be adjusted. For example, it can be achieved that the conductor elements preferably slightly contact an object arranged in the operation space 15. Thereby, the occurrence of free space, namely the air gap between the conductor elements 5, 7 and the tablet 10 can be reduced or minimized such that the accuracy of the solid fraction determination can be increased or optimized.

A thickness measuring unit (not shown in the Fig.) is adapted to measure a thickness of the tablet 10 positioned in the operation space 15, wherein the thickness measuring sensor comprises a distance capacitance sensor.

The first conductor element 5 and the second conductor element 7 is equipped with an insulating layer 19 towards the operation space 15 for minimizing effects of parasitic resistivity of the tablet 10 on the measurement. It may further help to increase the lifetime of the respective conductor element 5, 7. Also, it may help to prevent contamination of the tablet 10. Still further, it may prevent or reduce dust build up on the solid fraction sensor 17. Finally, it may also allow for easier cleaning of the solid fraction sensor 17 and particularly its conductor elements 5, 7.

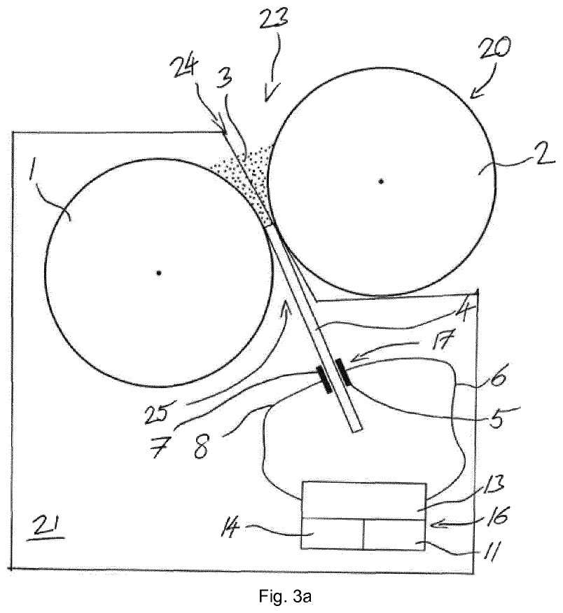

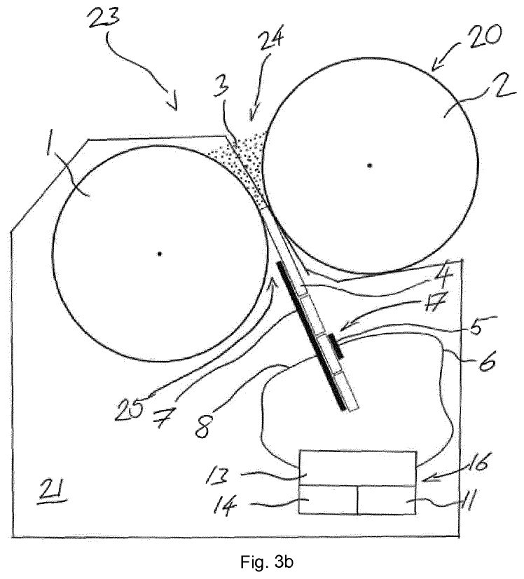

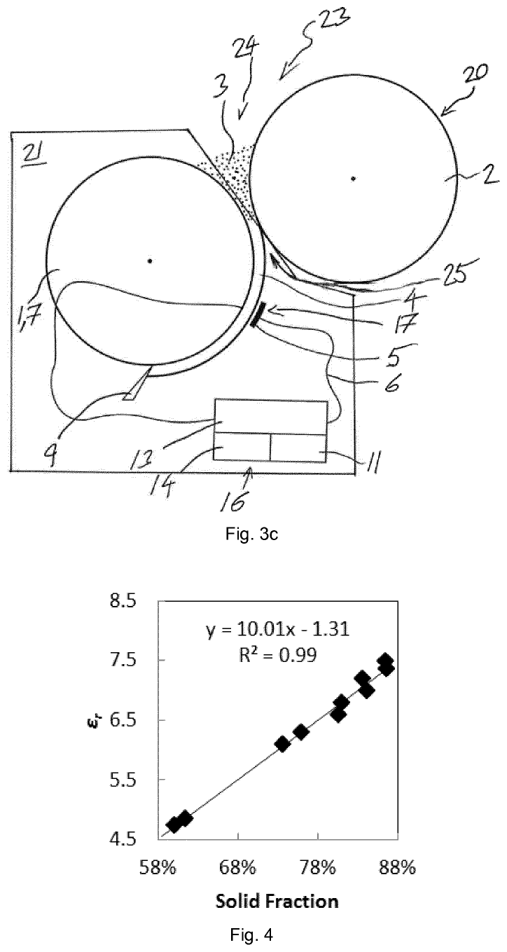

In FIG. 3a,b,c another intended application of the solid fraction sensor 17 in embodiments of pharmaceutical manufacturing installations is shown in the measurement of ribbons 4 prepared by roller compaction before they are milled. A typical roller compaction contains two rolls 1 and 2 which press powder 3 into a ribbon 4. The ribbon 4 is then milled into granules. The solid fraction of the ribbon 4 influences both hardness and size of the granules. It is therefore highly relevant to the bioavailability of the final pharmaceutical products via dissolution and disintegration characteristics. As revealed in all FIG. 3a,b,c, an overall pharmaceutical manufacturing installation 23 comprises a processing equipment 21 arranged to provide a pharmaceutical substance 4 in a solid state and a solid fraction sensor 17. Part of this processing equipment 21 is additionally a first conductor element 5, a second conductor element 7, an operation space 15, an energy source 13 arranged to generate an electric field in the operation space 15 by means of the first conductor element 5 and the second conductor element 7, and a controller 11 adapted to determine a capacitance between the first and second conductor element 5, 7 with the pharmaceutical substance 4 in the solid state located in the operation space 15, wherein the solid fraction sensor 17 is arranged to receive the pharmaceutical substance 4 in its solid state into the operation space 15 by the processing equipment 21. Through a powder inlet zone 24 powder 3 is fed to the processing equipment 21.

In FIG. 3a,b,c a possible implementation of the solid fraction sensor 17 within the roller compactor 20 is outlined. In an ideal case, a representative sample of ribbon 4 is produced without being stuck or keyed to any of the rolls 1, 2. In such circumstances, a similar solid fraction sensor 17 to one shown in FIG. 2 can be used and the ribbon 4 can be fed between the electrodes 5, 7 as shown in FIG. 3a.

In practice, the ribbons 4 may not always be strong enough and break. In such cases, the ground electrode 7 can be extended and serve as a support 26, as shown in FIG. 3b. Alternatively, a mechanical support to collect and guide the ribbon 4 can be added to the design, with the electrode 7 implemented within such support. The sensing area will be defined by the solid fraction sensor electrode 5.

When a collar is applied to the fixed roll 1, the ribbons 4 have a strong tendency to remain keyed to the fixed roll 1 and have to be scrapped off by a scraper 9 as shown in FIG. 3c. In such cases, the fixed roll 1 can be used as electrode 7 and the solid fraction sensor requires only one custom-made electrode 5. The sensing area will be again defined by the sensor electrode 5. In this case, the sensor electrode 5 may be curved to limit the inhomogeneity in the generated electric field.

In all cases, the solid fraction sensor 17 can be connected as a floating sensor (with ground electrode floating) or as a grounded sensor (with ground electrode grounded). When the ground electrode is connected as a floating electrode, one of the electrodes can be used for the excitation and another for the read-out. When ground electrode is grounded, the setup requires a switch (not shown) to allow for use of the sensor electrode for both excitation and read-out. The latter is practically useful for the cases described in FIGS. 3b and 3c. Here either the support or the roll should be grounded to minimize the parasitic capacitive and resistive signals from the machinery and other external disturbances.

FIG. 4 shows an example of a calibration curve in which pairs of permittivity and corresponding solid fraction ratio of a reference pharmaceutical substance are displayed. In particular, in the example calibration curve, a calibration obtained at uniform operating conditions on tablets with different thickness after thickness correction is shown.

This description and the accompanying drawings that illustrate aspects and embodiments of the present invention should not be taken as limiting—the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1-39. (canceled)

40. A pharmaceutical manufacturing installation comprising:
a processing equipment arranged to provide a pharmaceutical substance in a solid state; and
a solid fraction sensor,
wherein the sensor has a first conductor element, a second conductor element, an operation space, an energy source arranged to generate an electric field in the operation space by means of the first conductor element and the second conductor element, and a controller adapted to determine a capacitance between the first and second conductor elements with the pharmaceutical substance in the solid state located in the operation space, and
wherein the solid fraction sensor is arranged to receive the pharmaceutical substance in its solid state into the operation space by the processing equipment.

41. The pharmaceutical manufacturing installation of claim 40, wherein the processing equipment comprises a punch arrangement adapted to generate a tablet of the pharmaceutical substance.

42. The pharmaceutical manufacturing installation of claim 40, wherein one of the first conductor element and the second conductor element is a part of the punch arrangement of the processing equipment or of a tablet chute of the processing equipment.

43. The pharmaceutical manufacturing installation of claim 40, wherein
the processing equipment comprises two rotatable rolls and a compacting space in between the rolls,
the compacting space has a powder inlet zone and a ribbon outlet zone, and
the solid fraction sensor is arranged adjacent to the ribbon outlet zone of the compacting space such that a ribbon generated by the two rolls exiting the compacting space is forwarded into the operation space of the solid fraction sensor.

44. The pharmaceutical manufacturing installation of claim 43, wherein
the second conductor element is formed as a ribbon support adapted to guide a ribbon exiting the compacting space, or
one of the two rolls is the second conductor element, and the first conductor element preferably is bent to correspond to the outer surface of the one of the two rolls.

45. The pharmaceutical manufacturing installation of claim 40, wherein the operation space of the solid fraction sensor is a gap separating the first conductor element and the second conductor element.

46. The pharmaceutical manufacturing installation of claim 40, wherein
the controller of the solid fraction sensor comprises calibration data of a reference pharmaceutical substance having the essentially same dielectric properties as the pharmaceutical substance in its solid state,
the calibration data of the controller of the solid fraction sensor comprises composition data about the composition of the reference pharmaceutical substance and thickness data about the thickness of the reference pharmaceutical substance,
the controller of the solid fraction sensor is adapted to convert the calibration data and the determined capacitance into solid fraction data of the pharmaceutical substance in its solid state, and
the controller is adapted to generate a solid fraction signal representing the solid fraction data.

47. The pharmaceutical manufacturing installation of claim 40, wherein the energy source of the solid fraction sensor is connected to at least one of the first conductor element and the second conductor element.

48. The pharmaceutical manufacturing installation of claim 40, wherein the controller of the solid fraction sensor is adapted to adjust a strength of the electric field in the operation space.

49. The pharmaceutical manufacturing installation of claim 46, wherein
the controller of the solid fraction sensor has a data storage in which the calibration data is stored, and/or
the calibration data comprises permittivity of the reference pharmaceutical substance and a solid fraction ratio of the reference pharmaceutical substance, wherein the calibration data preferably comprises pairs of permittivity and corresponding solid fraction ratio.

50. The pharmaceutical manufacturing installation of claim 40, wherein the first conductor element of the solid fraction sensor and the second conductor element of the solid fraction sensor are metallic and plate-like shaped.

51. The pharmaceutical manufacturing installation of claim 40, wherein the controller of the solid fraction sensor is adapted to determine the capacitance by a capacitance-to-digital conversion, wherein the controller of the solid fraction sensor preferably is adapted to apply sigma-delta modulation to determine the capacitance.

52. The pharmaceutical manufacturing installation of claim 40, wherein
the controller of the solid fraction sensor is adapted to measure a discharge time and to determine the capacitance by using the measured discharge time, and/or
a charge-balancing circuit is used to measure the capacitance.

53. The pharmaceutical manufacturing installation of claim 40, further comprising:
a displacement structure, wherein at least one of the first conductor element of the solid fraction sensor and the second conductor element of the solid fraction sensor is mounted to the displacement structure such that the first conductor element of the solid fraction sensor and the second conductor element of the solid fraction sensor are movable relative to each other; and/or
a thickness measuring unit adapted to measure a thickness of the pharmaceutical substance in its solid state, preferably, when positioned in the operation space of the solid fraction sensor, wherein the thickness measuring sensor preferably comprises a distance capacitance sensor.

54. The pharmaceutical manufacturing installation of claim 40, wherein at least one of the first conductor element of the solid fraction sensor and the second conductor element of the solid fraction sensor is equipped with an insulating layer towards the operation space.

55. A method of manufacturing a pharmaceutical product, comprising:
providing a pharmaceutical substance in a solid state;
positioning the pharmaceutical substance in its solid state in an operation space in which an electric field is generated by means of a first conductor element and a second conductor element;
determining a capacitance of the pharmaceutical substance in its solid state located in the operation space; and
converting the determined capacitance together with information about a composition of a reference pharmaceutical substance having the essentially same dielectric properties as the pharmaceutical substance in its solid state and about a thickness of the reference pharmaceutical substance into a solid fraction of the pharmaceutical substance in its solid state.

56. The method of claim 55, wherein the pharmaceutical substance in its solid state is bounded, or the pharmaceutical substance in its solid state is unbounded.

57. The method of claim 55, further comprising:
adjusting a strength of the electric field in the operation space; and/or
measuring a thickness of the target pharmaceutical sample positioned in the operation space.

58. The method of claim 55, wherein
the information about the composition of the reference pharmaceutical substance comprises permittivity of the reference pharmaceutical substance and a solid fraction ratio of the reference pharmaceutical substance,
the information about the composition of the reference pharmaceutical substance and about the thickness of the reference pharmaceutical substance preferably comprises pairs of permittivity and corresponding solid fraction ratio, and
the information about the composition of the reference pharmaceutical substance and about the thickness of the reference pharmaceutical substance preferably is a calibration curve.

59. The method of claim 55, wherein
the capacitance is determined by a capacitance-to-digital conversion, wherein preferably a sigma-delta modulation is applied to determine the capacitance, and/or
a discharge time is measured and the capacitance is determined by using the measured discharge time, and/or
a charge-balancing circuit is used to measure the capacitance.

60. The method of claim 55, wherein the at least one of the first conductor element and the second conductor element is displaced to adjust the operation space.

61. The method of claim 55, further comprising:
positioning the pharmaceutical substance in its solid state in a further operation space of the solid fraction sensor or a further solid fraction sensor, having a further first conductor element, a further second conductor element, the further operation space and a further energy source arranged to generate an electric field in the further operation space by means of the further first conductor element and the further second conductor element;
determining a further capacitance of the pharmaceutical substance in its solid state located in the further operation space;
converting the determined further capacitance together with the information about the composition of the reference pharmaceutical substance and about the thickness of the reference pharmaceutical substance into a further solid fraction of the pharmaceutical substance in its solid state; and
determining a solid fraction distribution of the solid fraction of the pharmaceutical substance in its solid state and the further solid fraction of the pharmaceutical substance in its solid state.

62. The method of claim 55, further comprising adjusting a distance between the first conductor element and the second conductor element such that the first conductor element and the second conductor element contact the pharmaceutical substance in its solid state when positioned in the operation space.

63. The method of claim 55, further comprising:
positioning the pharmaceutical substance in its solid state in a reference operation space of a reference solid fraction sensor having a reference first conductor element, a reference second conductor element, the reference operation space and a reference energy source arranged to generate an electric field in the reference operation space by means of the reference first conductor element and the reference second conductor element;
determining a reference capacitance of the pharmaceutical substance in its solid state located in the reference operation space;
converting the determined reference capacitance together with the information about the composition of the reference pharmaceutical substance and about the thickness of the reference pharmaceutical substance into a reference solid fraction of the pharmaceutical substance in its solid state; and
comparing the solid fraction of the pharmaceutical substance in its solid state to the reference solid fraction of the pharmaceutical substance in its solid state.
